(19) United States
(12) Patent Application Publication
(10) Pub. No.: US 2022/0076830 A1
(43) Pub. Date: Mar. 10, 2022

(54) SIDE EFFECT AND SYMPTOM RELIEF

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/239,777

(22) Filed: Apr. 26, 2021

Related U.S. Application Data
(63) Continuation of application No. PCT/IB2019/058925, filed on Oct. 18, 2019.
(60) Provisional application No. 62/750,146, filed on Oct. 24, 2018.

(51) Int. Cl. G16H 50/20; G16H 40/63; G16H 20/10

(52) U.S. Cl. CPC G16H 50/20; G16H 20/10; G16H 40/63

(57) ABSTRACT

A method and system for the relief from side effects associated with the consumption of cannabis, cannabis extracts, cannabis products, or isolated or synthetic forms thereof. The apparatus comprising a sealed vessel for containing the composition, which may be in the form of a liquid, powder, tablet, topical lotion, tincture, or pressurized vapor. In some embodiments, the container is designed to be easily accessible to those under the influence of THC, provides evidence of prior use or tampering, includes child and/or pet safety measures, or other security considerations. In some embodiments, the composition may be inhaled, drunk, swallowed, or administered sublingually, topically, transdermal, oracularly, nasally, peroral, transmucosal, injection or nanoneedle, for example. In some embodiments, the composition may be designed to provide relief from the side effects of a cultivar of cannabis (e.g. Charlotte's Web, Northern Lights, etc.). In some embodiments, the composition and apparatus are sold as a kit along with various other cannabis side effect remedies, or cannabis, or a cannabis product.

### CROSS-REFERENCE TO RELATED APPLICATIONS

The present patent application is a continuation of International Application No. PCT/IB2019/058925 filed Oct. 18, 2019, which claims the priority benefit of U.S. provisional patent application 62/750,146 filed Oct. 24, 2018, the disclosures of which are incorporated by reference herein.

### BACKGROUND OF THE INVENTION

### 1. Field of the Disclosure

The present disclosure is generally related to relieving the side effects of cannabis.

### 2. Description of the Related Art

Cannabis is a genus belonging to the family of cannabaceae. Three common species include Cannabis sativa, Cannabis indica, and Cannabis ruderalis. Cannabis has a long history being used for medicinal, therapeutic, and recreational purposes. Historical delivery methods for cannabis have involved smoking (e.g., combusting) the dried cannabis plant material. Alternative delivery methods such as ingesting typically require extracts of the cannabis biomass, such as cannabis concentrates or cannabis oils. Often, cannabis extracts are formulated using any convenient pharmacologically or food-grade acceptable diluents, carriers or excipients to produce a composition, which collectively may be known as cannabis derivative products or cannabis products. These may for example include cannabis topicals, edibles, or vaping products.

Cannabis contains a unique class of terpeno-phenolic compounds known as cannabinoids or phytocannabinoids. The principle cannabinoids present in cannabis are the delta-9-tetrahydrocannabinolic acid (THCA) and cannabidiolic acid (CBDA). The THCA does not have its own psychoactive properties as is, but may be decarboxylated to delta-9-tetrahydrocannabinol (THC), which is a potent psychoactive cannabinoid. The neutral form of CBDA is cannabidiol (CBD), which is a major cannabinoid substituent in hemp cannabis. CBD is non-psychoactive and is widely known to have therapeutic potential for a variety of medical conditions. The proportion of cannabinoids in the plant may vary from species to species, as well as vary within the same species at different times and seasons. Furthermore, the proportion of cannabinoids in a plant may further depend upon soil, climate, and harvesting time and methods. Thus, based on the proportion of the cannabinoids present in a plant variety, the psychoactive and medicinal effects obtained from different plant varieties may vary. Such variance is further exacerbated by the presence of certain terpenoid or phenolic compounds that may also have pharmacological activity.

THC and CBD have been found to have opposing neuropharmacological actions. THC is known to potentially produce psychotic-like and anxiogenic effects in humans, depending on the dose delivered. In contrast, CBD may have anti-psychotic properties, is anxiolytic, and may be neuroprotective in humans. THC is a partial agonist, whereas CBD is an antagonist at CB1 and CB2 receptors. CBD has also been suggested to inhibit the reuptake of the endogenous cannabinoid, anandamide.

However, the relative THC/CBD ratio of cannabis varies greatly. Levels of CBD can range from virtually none to up to 40%. Higher levels of THC are found in certain cultivars and in cross-bred strains, which are increasingly dominating the illicit drug market. In addition to effects on psychotic symptoms and anxiety, THC and CBD may have opposing effects in the processes involved in addiction. The reinforcing effects of THC have been repeatedly shown. Synthetic THC produces conditioned place preference in rats and decreases the threshold for intercranial self-stimulation in animal studies. CBD on its own is not acutely reinforcing in rats. However, CBD has been shown to reverse the conditioned place preference effect induced by THC in CBD to THC ratios of 1:1 and 1:10, which suggests that CBD may modulate the reinforcing effects of THC. CBD has also been suggested to have a function in the modulation of addictive behavior. Preclinical studies have shown that acute administration of CBD can enhance extinction of both cocaine and amphetamine conditioned place preference. CBD has also been found to attenuate the reinstatement of opioid seeking in rats. Given the opposing neuropharmacological actions of THC and CBD, and the capacity of CBD to modulate the acute reinforcing effects of THC in rats, it is hypothesized that CBD may also counteract some of the reinforcing effects of THC in humans.

THC has been the primary focus of cannabis research since 1964, when Raphael Mechoulam isolated and synthesized it. More recently, the synergistic contributions of CBD to cannabis pharmacology and analgesia have been scientifically demonstrated. Other phytocannabinoids (e.g., tetrahydrocannabivarin, cannabigerol and cannabichromene) exert additional effects of therapeutic interest. Innovative conventional plant breeding has yielded cannabis chemotypes expressing high titres of each component for future study. Additionally, another echelon of phytotherapeutic agents contributes to the effects of cannabis, the cannabis terpenoids and terpenes such as limonene, myrcene, α-pinene, linalool, β-caryophyllene, caryophyllene oxide, nerolidol, and phytol. Terpenoids share a precursor with phytocannabinoids and are all flavor and fragrance components common to human diets that have been designated “Generally Recognized as Safe” by the U.S. Food and Drug Administration and other regulatory agencies. Terpenoids are quite potent and can affect animal, including human, behavior when inhaled from ambient air at serum levels in the single digits ng-mL. Terpenoids display unique therapeutic effects that may contribute meaningfully to the entourage of effects of cannabis-based medicinal extracts. Particular focus may be placed on phytocannabinoid-terpenoid interactions that could produce synergy with respect to treatment of pain, inflammation, depression, anxiety, addiction, epilepsy, cancer, fungal, and bacterial infections such as methicillin-resistant Staphylococcus aureus. Scientific evidence has presented non-cannabinoid plant components as putative antidotes to intoxicating effects of THC that could increase its therapeutic index.

The general knowledge on cannabis toxicity has improved, but quantitative data are still lacking. Little is known about the somatic complications associated with cannabis exposure. Available data rest upon case reports and only a few studies have been conducted in this field. Psychiatric disorders related to cannabis exposure are somehow still controversial. Methods for utilizing the entourage effects in a composition and apparatus have been proposed. Phytocannabinoid-terpenoid synergy, if proven, increases the likelihood that an extensive pipeline of new therapeutic products is possible from this venerable plant.

There exists a need for a cannabinoid and terpenoid based treatment for the onset of side effects related to the consumption of cannabis, especially cannabis with a relatively high ratio of THC to other cannabinoids such as CBD.

The present invention is concerned with a composition and apparatus capable of providing relief from side effects and symptoms associated with the consumption of cannabis and/or its constituent compounds, namely THC. Side effects and symptoms associated with the consumption of cannabis and/or its constituent components include, for example, red eyes, poor muscle coordination, delayed reaction times, increased appetite, mood swings, anxiety, panic, hallucinations, headache, dizziness, drowsiness, nausea, and paranoia.

### BRIEF DESCRIPTIONS OF THE DRAWINGS

FIG. 1 illustrates an exemplary network environment in which a system for providing cannabis side effect and symptom relief may be implemented.

FIG. 2 is a flowchart illustrating an exemplary method for providing cannabis side effect and symptom relief.

FIG. 3 is a flowchart illustrating an exemplary method of profiling a user for cannabis side effect and symptom relief.

FIG. 4 is a table illustrating exemplary remedies that may be included in a remedy database.

FIG. 5 is a flowchart illustrating an exemplary method for monitoring cannabis side effects and symptoms.

FIG. 6 is a flowchart illustrating an exemplary method of making recommendation for cannabis side effect and symptom relief.

FIG. 7 is a flowchart illustrating an exemplary method for tracking treatments for cannabis side effect and symptom relief.

FIG. 8 is a flowchart illustrating an exemplary method for tracking user data via a wearable device.

FIG. 9 is a table illustrating exemplary remedies in the remedy network database.

FIG. 10 is a flowchart illustrating an exemplary method for managing cannabis side effects and symptom relief.

### DETAILED DESCRIPTION

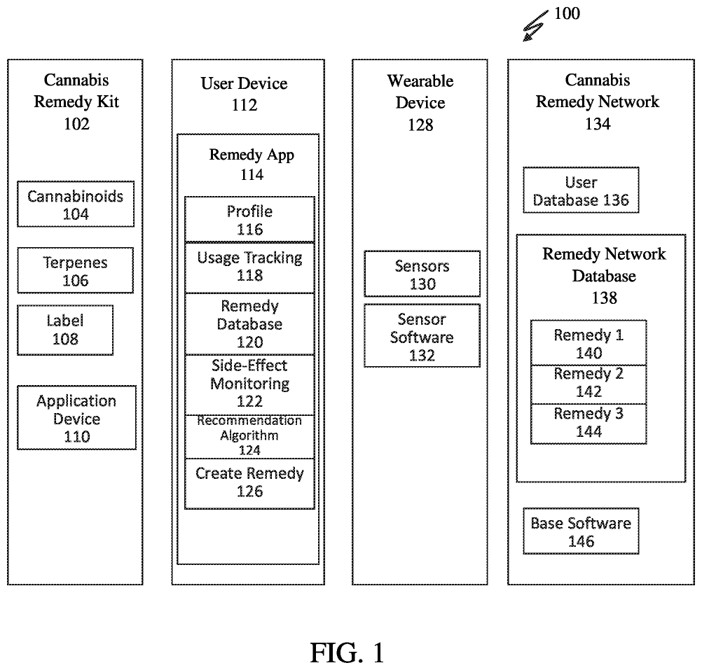

Embodiments of the present disclosure include systems and methods for providing cannabis side effect and symptom relief. FIG. 1 is a block diagram illustrating an exemplary cannabis remedy system 100 for providing relief to side effects and symptoms that result from cannabis usage. The cannabis remedy system 100 may include a cannabis remedy kit 102, a user device 112, a wearable device 128, and a cannabis remedy network 134. The cannabis remedy kit 102 may include cannabinoids 104, terpenes 106, a label 108, and application devices 110. The cannabinoids 104 may be a mixture of specific cannabinoids such as CBD that is formulated to provide the consumer with relief from the side effects of cannabis use. Some examples of side effects from cannabis include anxiety, paranoia, panic, and discomfort. The terpenes 106 may include a selection of specific terpenes formulated to provide the consumer with relief from the side effects of cannabis use. The formulation of certain terpenes may be more effective for specific strains of cannabis than others. The label 108 may contain warnings, disclaimers, a list of items in the kit 102, a description of intended use, and directions for downloading the cannabis remedy application 114 on a user device 112. The application devices 110 for applying or consuming the cannabis remedy may be fillable capsules, spray devices, vaporizers, topical creams, patches, quick dissolving oral strips, etc.

The cannabis remedy kit may include the user device 112 that is capable of downloading and executing the cannabis remedy application 114. The cannabis remedy application 114 may store user profile data and recommend a remedy based on type and amount of cannabis consumed and severity of symptoms. The cannabis remedy application 114 may include several software modules a user profile 116, a usage tracking 118, a remedy database 120, a side-effect monitoring 122, a recommendation algorithm, and a create remedy 126.

The user profile 116 contains the user data that may be analyzed by the recommendation algorithm 124 to determine a recommended remedy. The profile 116 may include data such as the users height, weight, age, sex, medical information, current medications, personality type (e.g., introvert or extrovert), cannabis tolerance (e.g. high, medium, or low), cannabis vehicle preferences (e.g. smoking, vaporizing, edibles and drinkables, topical treatments, etc.).

The usage tracking module 118 of the cannabis remedy application 114 may allow the user to accurately track their cannabis consumption. Based on the tracking, the recommendation algorithm module 124 may recommend a remedy for any side effects of cannabis.

The remedy database 120 is a database of remedies that the user may download from the cannabis remedy network 134. The remedy database 120 may include recommendations for specific treatment applications and specific therapeutic behaviors. Some examples of specific treatment applications are the administration of one or more cannabinoids or terpenes contained in the cannabis remedy kit 102 with a particular application device 110. Some examples of recommendations of specific therapeutic behaviors are drinking water, exercising, lying down, taking a bath, listening to relaxing music, calling a trusted friend, and seeking medical attention including telemedicine.

The side-effect monitoring module 122 cannabis remedy application 114 monitors the side-effects of cannabis of the user. Some examples of methods for side-effect monitoring 122 include tracking biometrics provided by a wearable device, tracking the users response time while performing certain tasks on the user device 112, and providing a questionnaire regarding their current experience of cannabis-related side-effects. The biometrics that the wearable device may provide include the heart rate, the heart rate variability, blood pressure, blood oxygen content, skin temperature, electroencephalograms (EEG) and electrocardiograms (ECG) activity, sleep patterns, etc. The tasks on the user device 112 may include performing a skill-based game, solving simple math problems, observing changes in scenery or sounds, etc.

The recommendation algorithm module 124 of the cannabis remedy application recommends the remedy best suited to the user based on the data from the user profile 116, usage tracking 118, and side-effect monitoring 122. In some cases, the recommendation algorithm 124 may not find a matching remedy. In such cases, the recommendation algorithm 124 requests addition remedies from the remedy network database 138 of the cannabis remedy network 134. Furthermore, the create remedy module 126 of the cannabis remedy application may allow the user to create a custom remedy, which may be uploaded to the cannabis remedy network 134 to allow other users to benefit from the created remedy.

The wearable device 128 of the cannabis remedy system 100 monitors the user's biometric data through a series of sensors. The wearable device 128 contains sensors 130 and a software module for the sensors 132. Any of several biometric sensors 130 may be used to monitor the user's biometric data such as heart rate, the heart rate variability, blood pressure, blood oxygen content, skin temperature, electroencephalograms (EEG) and electrocardiograms (ECG) activity, sleep patterns, etc. The sensor software module 132 on the wearable device 128 may be used to retrieve the sensor data from the sensors 130 of the wearable device 128 and send the sensor data to the user device 112 to monitor cannabis side-effects.

The cannabis remedy network 134 is a network in the cloud or internet, which contains and provides cannabis side-effects remedies to the cannabis remedy application 114 on the user device 112. The cannabis remedy network 134 allows users to upload cannabis remedies that were successful.

The cannabis remedy network contains a user database 136, a remedy network database 138, various remedies 140, 142, 144, and a base software 146. The user database 136 is a database of registered users of the cannabis remedy network 134 that may include each of the user's profile data. By registering with the cannabis remedy network 134, each user may download remedies from the remedy network database 138 or upload created remedies from the create remedy module 126 of the cannabis remedy application 114 to the remedy network database 138.

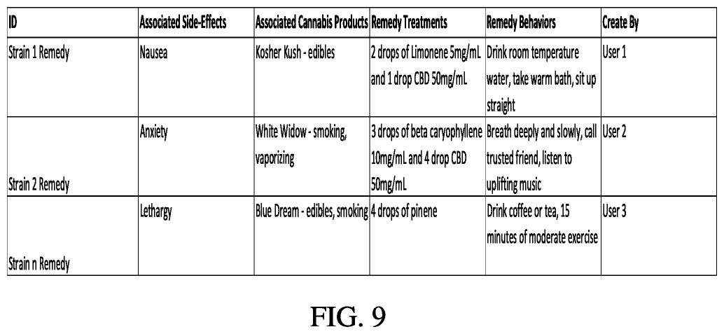

The remedy network database 138 is a database of remedies that a registered user may download from the cannabis remedy network. The remedies in this database 138 may include data regarding specific strains, side-effects, user attributes. The data may be used to match a user's cannabis side-effects with a recommended remedy. The remedy network data may include unlimited number of remedies 140, 142, and 144. Remedy 1 140 and remedy 2 142, and potential remedy n 144 are example remedies that will be further discussed in conjunction with FIG. 9.

The base software 146 of the cannabis remedy network 134 may allow the user to upload and download cannabis remedies from the remedy network database 138. The base software 146 may also match user data located in the user database 136 with the best remedy for cannabis side effects based on the recommendation algorithm 124 on the user device.

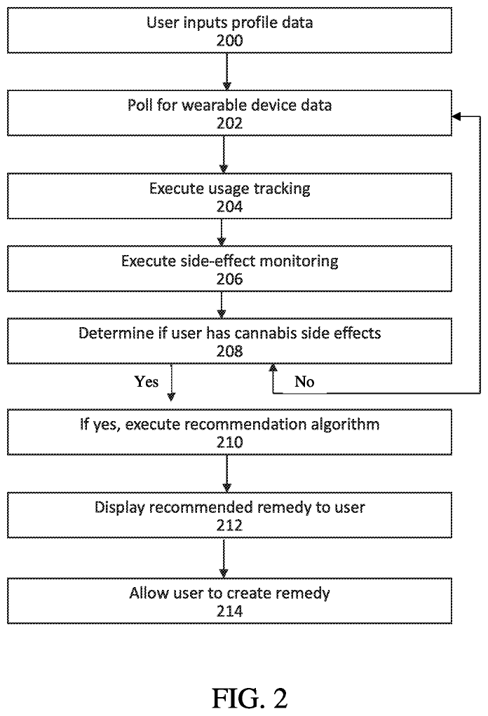

FIG. 2 is a flowchart illustrating an exemplary process of the cannabis remedy application 114. First a user inputs the user's profile data at step 200. The user profile data may include the user's height, weight, age, sex, medical information and medications, personality type (e.g., introvert or extrovert), cannabis tolerance (e.g. high, medium, or low), cannabis application preferences (e.g. smoking, vaporizing, edibles, drinkables, topical treatments, etc.). Next the cannabis remedy application 114 will poll the wearable device for wearable device sensor data at step 202. The sage Tracking module will be executed to track the user data from sensor or user input at step 204. The side-effect monitoring module will be executed at step 206. The cannabis remedy application 114 may then determine if the user has cannabis side-effects based on the data collected by the usage tracking module 118 and the data collected by the side-effect monitoring module 122 at step 208. In an embodiment, the user may self-report cannabis side effects to manually trigger the recommendation algorithm 124. If the application 114 determines that the user has cannabis side effects, the application 114 will execute the recommendation algorithm module 124 at step 210. The application 114 may then receive and display to the user the recommendation from the recommendation algorithm 124 at step 212. In an embodiment, the user may be allowed to create and upload a remedy that was successful in managing the cannabis side effects to the application 114 using the create remedy module 126 at step 214.

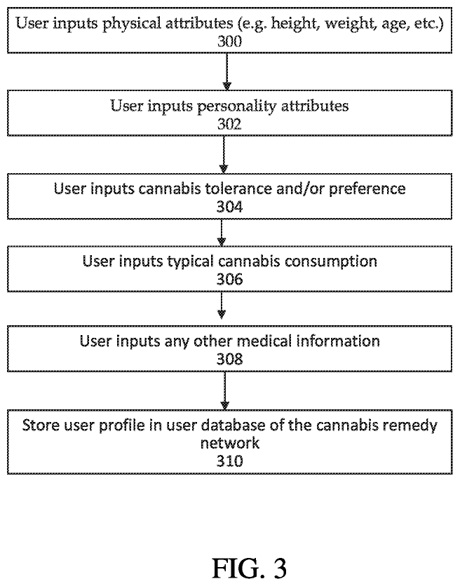

FIG. 3 is a flowchart illustrating an exemplary process of the profile module 116. First a user inputs physical attributes such as the user's height, weight, age at step 300. Then the user may input personality attributes such as introvert and extrovert personalities at step 302. The user may then input their preferences at step 304. Examples of preferences include the user's level of cannabis tolerance (high, medium, low), the user's preferences in types of cannabis plants such as sativa, indica, or hybrid plants, and the user's preference in the method of use such as smoking, vaporizing, and edibles. The user may then input typical daily/weekly/monthly cannabis consumption at step 306. Next, the user may input any medical information such as diseases, ailments and current and past medications at step 308. The profile module 116 may then store all the inputted profile data into the user database 136 of the cannabis remedy network 134 at step 310.

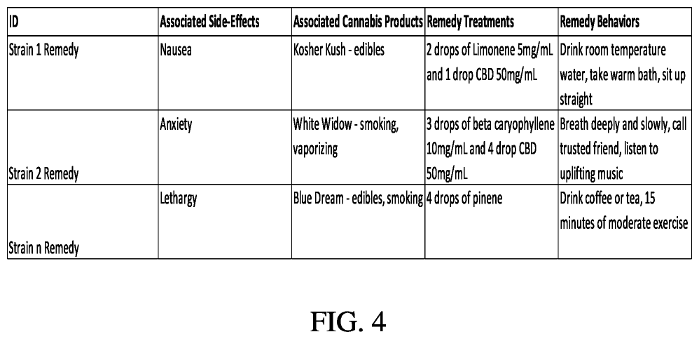

FIG. 4 is a table illustrating exemplary remedies in the remedy database 120

The remedy database 120 is a database of remedies that the user has downloaded from the remedy network database 138 of the cannabis remedy network 134. The remedy database 120 identifies the name of the remedy, the associated side-effects, the associated cannabis products, the recommended treatments, and the recommended behavior, as seen in FIG. 4. FIG. 4 identifies three exemplar remedies that may be downloaded from the remedy network database 138 onto the user device 112 via the remedy database module 120 of the cannabis application 114. Remedy 1 is a remedy for the associated side effect of nausea that is associated with eating the cannabis product named “Kosher Kush.” The recommended remedy may be two drops of limonene and one drop of CBD at 50 mg/L concentration. The recommended behavior may be to drink room temperature water, take a warm bath, and sit up straight. Remedy 2 is a remedy for the associated side effect of anxiety that is associated with smoking or vaporizing the cannabis product named “White Widow.” The recommended remedy may be three drops of beta caryophyllene 10 mg/ml and four drops of CBD at 50 mg/L concentration. The recommended behavior may be to breathe deeply and slowly, call a trusted friend, and listen to uplifting music. Remedy 3 is a remedy for the associated side effect of lethargy that is associated with eating or smoking the cannabis product named “Blue Dream.” The recommended remedy may be four drops of pinene. The recommended behavior may be to drink coffee or tea and do 15 minutes of moderate exercise.

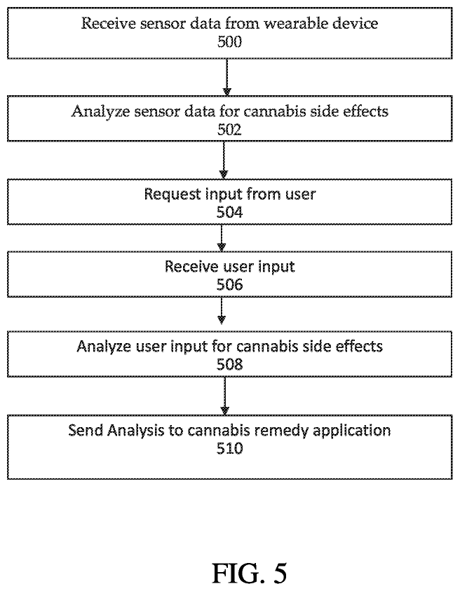

FIG. 5 is a flowchart illustrating an exemplary process of the side-effect monitoring module 122. First the side-effect monitoring module 122 of the cannabis remedy application 114 receives sensor data from a user's wearable device 128 at step 500. Sensor data from wearable device may include data for accelerometer and movement, heart rate, temperature, and other biometrics. Next, the side-effect monitoring module 122 may analyze the sensor data for cannabis side effects at 502. Cannabis side effects may be revealed by analyzing any increase or decrease from a baseline data for movement heart rate, temperature, or other abnormal biometric data. The side-effect monitoring module 122 may then request input from user at step 504. The requested user input may include direct observation of the side effects such as anxiety, lethargy or indirect observations for side effects such as observations of the performance of certain tasks and interactive game. Next, the side-effect monitoring module 122 may receive user input at step 506. The side-effect monitoring module 122 may analyze user input for one or more cannabis side effects, which can then be matched with a specific cannabis remedy at step 508. The side-effect monitoring module 122 may then send any analysis to the remaining modules in the cannabis remedy application 114 for further processing at step 510.

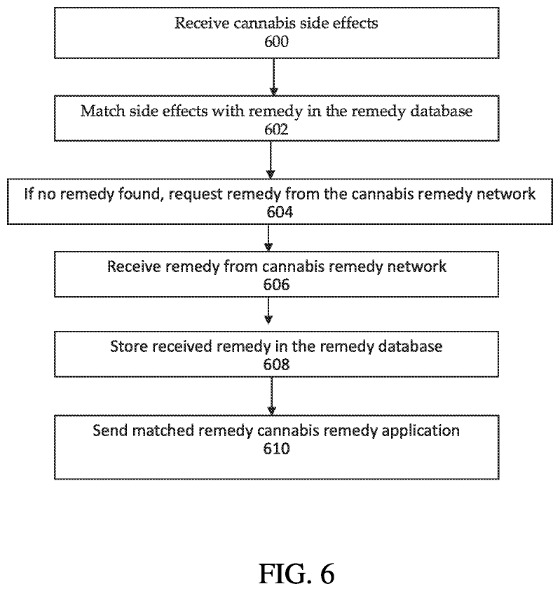

FIG. 6 is a flowchart illustrating an exemplary process of the recommendation algorithm module 124. First, the recommendation algorithm module 124 of the cannabis remedy application 114 may receive a report of cannabis side effects such as anxiety or lethargy at step 600. The recommendation algorithm module 124 may then match the identified side effects with a remedy in the remedy database 120 at step 602. If no remedy found, the recommendation algorithm module 124 may then request for a remedy from the cannabis remedy network 134 at step 604. The application module 124 may then receive one or more remedies from cannabis remedy network 134 at step 606 and store the received remedy in the remedy database 120 at step 608. The recommendation algorithm module 124 may then send matched remedy to the remaining modules of the cannabis remedy application 114 for further processing at step 610

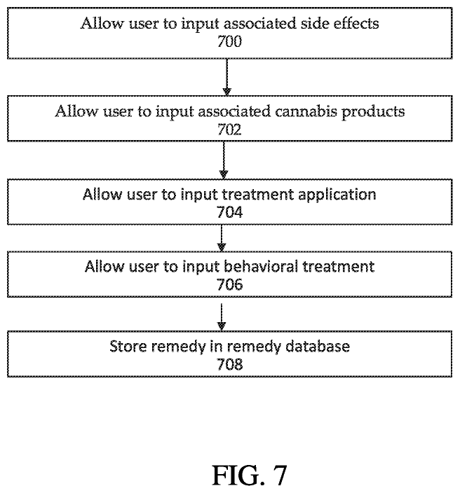

FIG. 7 is a flowchart illustrating an exemplary process of the create remedy module 126. First the create remedy module 126 may allow the user to input associated side-effects at step 700. Then the module may allow user to input associated cannabis products at step 702. The create remedy module 126 may then allow the user to input method of treatment and application, such as inhaling terpenes, cannabinoids, at step 704. The user may further input behavioral treatments such as listening to relaxing music and taking a warm bath, at step 706. The create remedy module 126 may then store the remedy in the remedy database 120 at step 708. Finally, the create remedy module may send the created remedy to the cannabis remedy network 134 at step 710.

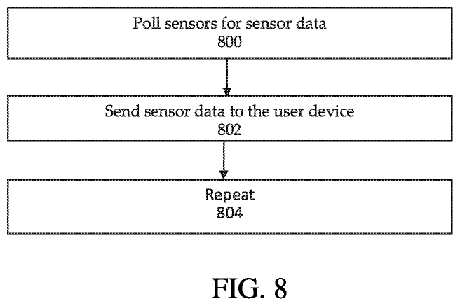

FIG. 8 is a flowchart illustrating an exemplary process of a sensor software 132 for the wearable device 128. First the software 132 may poll sensors located in the wearable device for data at step 800. The sensor software may then send the sensor data to the user device 112 at step 802. The process of polling for sensor data on the wearable device 128 and transmitting this data to the user device 112 is continually repeated at step 804.

FIG. 9 is a table illustrating exemplary remedies in the remedy network database 138 of the cannabis remedy network 134. The cannabis network database 138 maintains a database of remedies that may be downloaded by the user onto the remedy database 120 of the user device 112. FIG. 9 shows that the remedy network database 138 identifies the name of the remedy, the associated side-effects, the associated cannabis products, the recommended treatments, the recommended behavior, and user who created the remedy. FIG. 4 identifies three exemplar remedies. Remedy 1 is a remedy for the associated side effect of nausea that is associated with eating the cannabis product named “Kosher Kush.” The recommended remedy may be two drops of limonene and one drop of CBD at 50 mg/L concentration. The recommended behavior may be to drink room temperature water, take a warm bath, and sit up straight. The exemplar user “User 1” created this remedy. Remedy 2 is a remedy for the associated side effect of anxiety that is associated with smoking or vaporizing the cannabis product named “White Widow.” The recommended remedy may be three drops of beta caryophyllene 10 mg/ml and four drops of CBD at 50 mg/L concentration. The recommended behavior may be to breathe deeply and slowly, call a trusted friend, and listen to uplifting music. The exemplar user “User 2” created this remedy. Remedy 3 is a remedy for the associated side effect of lethargy that is associated with eating or smoking the cannabis product named “Blue Dream.” The recommended remedy may be four drops of pinene. The recommended behavior may be to drink coffee or tea and do 15 minutes of moderate exercise. The exemplar user “User 3” created this remedy.

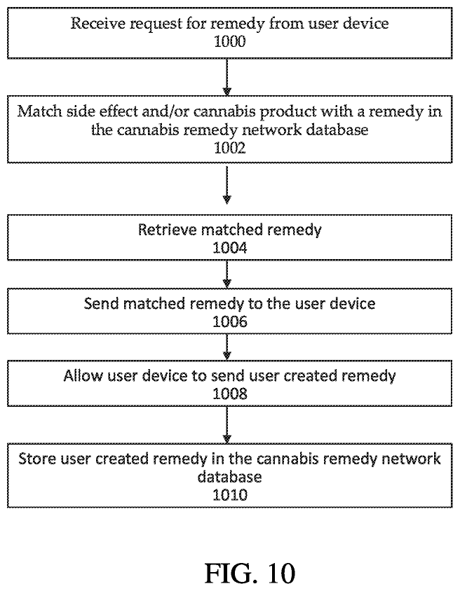

FIG. 10 is a flowchart illustrating an exemplary process of the base software 146 of the cannabis remedy network 134. First, the base software 146 may receive a request for remedy from the user device 112 at step 1000. Then the software 146 may match the side effect and cannabis product with a remedy in the cannabis remedy network database 138 at step 1002. The base software 146 may then retrieve the matched remedy at step 1004 and send the matched remedy to the user device 112 at step 1006. The base software 146 may allow the user device 112 to send user-created remedy to the cannabis remedy network 134 at step 1008, where the user-created remedy may be stored in the cannabis remedy network database 138 at step 1010.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claims.

What is claimed is:

1. A method for relieving the side effects of cannabis consumption, the method comprising:
storing a user profile in memory, the user profile including one or more attributes regarding a user;
storing information regarding a remedy kit that includes a set of available remedies;
monitoring the user after consumption of a cannabis product via one or more sensors associated with a wearable device;
identifying that a current set of sensor data is indicative of one or more physical effects;
generating a recommendation that includes at least one remedy corresponding to the identified physical effects, wherein the generated recommendation is based on the stored information regarding the set of available remedies in the remedy kit.

2. The method of claim 1, wherein the remedy kit further includes an application device, and wherein the generated recommendation includes directions for administering the at least one remedy.

3. The method of claim 2, wherein the application device includes at least one of fillable capsules, spray devices, vaporizers, topical creams, patches, and quick dissolving oral strips, wherein generating the recommendation further includes selecting the application device for administering the at least one remedy.

4. The method of claim 1, further comprising receiving data regarding the one or more user attributes via a downloadable application.

5. The method of claim 1, further comprising monitoring the user by analyzing one or more responses to at least one of questionnaire, skill-based game, series of problem-solving exercises, changes in scenery, and changes in sounds, wherein identifying the physical effects is further based on the analyzed responses.

6. The method of claim 1, further comprising polling the sensors, wherein the sensors track at least one of heart rate, the heart rate variability, blood pressure, blood-oxygen content, skin temperature, electroencephalograms activity, electrocardiograms activity, and sleep patterns.

7. The method of claim 1, wherein identifying the physical effects includes comparing the current set of sensor data to a predefined baseline, wherein the comparison indicates an increase or decrease from the predefined baseline.

8. The method of claim 7, wherein the recommendation is generated when the increase or decrease exceeds a range from the predefined baseline.

9. The method of claim 7, wherein the predefined baseline is specific to the user.

10. The method of claim 1, further comprising of receiving data regarding at least one new remedy added to the remedy kit, the data received via a user interface.

11. The method of claim 10, further comprising updating a remedy database to include the at least one new remedy.

12. The method of claim 1, further comprising uploading the user profile to a cloud server.

13. The method of claim 1, further comprising updating the user profile based on the identified physical effects and the generated recommendation.

14. The method of claim 1, further comprising monitoring the user after administering the at least one remedy to confirm whether the identified physical effects continue to be exhibited.

15. The method of claim 14, further comprising generating a next recommendation based on whether the identified physical effects continue to be exhibited following administration of the at least one remedy.

16. The method of claim 1, further comprising downloading information over a communication network regarding the at least one remedy from a remote remedy database.

17. The method of claim 1, wherein the recommendation is generated based on the user profile.

18. A system for relieving the side effects of cannabis consumption, the system comprising:
memory that stores a user profile and information regarding a remedy kit, the user profile including one or more attributes regarding a user and the remedy kit including a set of available remedies;
a wearable device including one or more sensors that monitor the user after consumption of a cannabis product; and
a processor that executes instructions stored in memory, wherein the processor executes the instructions to identify that a current set of sensor data is indicative of one or more physical effects; and
a user interface that displays a generated recommendation that includes at least one remedy corresponding to the identified physical effects, wherein the generated recommendation is based on the stored information regarding the set of available remedies in the remedy kit.

19. A non-transitory, computer-readable storage medium, having embodied thereon a program executable by a processor to perform a method for relieving the side effects of cannabis consumption, the method comprising:
storing a user profile in memory and information regarding a remedy kit, the user profile including one or more attributes regarding a user and the remedy kit including a set of available remedies;
monitoring the user after consumption of a cannabis product via one or more sensors associated with a wearable device;
identifying that a current set of sensor data is indicative of one or more physical effects;
generating a recommendation that includes at least one remedy corresponding to the identified physical effects, wherein the generated recommendation is based on the stored information regarding the set of available remedies in the remedy k
